(19) United States
(12) Patent Application Publication
(10) Pub. No.: US 2005/0239743 A1
(43) Pub. Date: Oct. 27, 2005

(54) Compositions and methods to inhibit restenosis

(75) Inventors: Eliezer Zomer, Newton, MA (US); David Platt, Newton, MA (US); Anatole Klyosov, Newton, MA (US)

Correspondence Address: PERKINS, SMITH & COHEN LLP, ONE BEACON STREET, 30TH FLOOR, BOSTON, MA, 02108, US

(73) Assignee: Pro-Pharmaceuticals, Inc., Newton, MA (US)

(21) Appl. No.: 11/103,310

(22) Filed: Apr. 11, 2005

Related U.S. Application Data
(60) Provisional application No. 60/561,306, filed on Apr. 12, 2004.

(51) Int. Cl. A61K 38/16; A61K 31/736; A61K 31/7072; A61K 31/7076; A61K 38/21; A61K 31/4745

(52) U.S. Cl. 514/54; 424/649; 514/34; 514/102; 514/27; 514/283; 514/171; 514/46; 514/49; 514/410; 514/651; 424/85.7; 514/449; 514/492; 514/575; 514/251; 514/8

(57) ABSTRACT

Disclosed herein are compositions and methods comprising a polysaccharide in combination with a pharmaceutical agent wherein the composition can be used to prevent restenosis.

### RELATED APPLICATION

This application claims the benefit of and priority to U.S. Provisional application 60/561,306, filed Apr. 12, 2004.

### FIELD OF THE INVENTION

The present invention relates to compositions and methods for inhibiting vascular intimal hyperplasia, better known as restenosis.

### BACKGROUND OF THE INVENTION

Stenosis means constriction or narrowing. A coronary artery that is constricted or narrowed is called stenosed. Buildup of fat, cholesterol and other substances over time may contribute to vessel stenosis.

One way to widen a coronary artery is through PTCA (balloon angioplasty). About one-third of patients who undergo PTCA have restenosis (renarrowing) of the widened segment within about six months of the procedure. Restenosed arteries may have to undergo another angioplasty. Currently researchers are trying to find ways to prevent restenosis such as by using stents. A stent is a wire mesh tube used to prop open an artery that can reduce the likelihood of restenosis.

Restenosis can also occur after a coronary artery bypass graft (CABG) operation. This type of heart surgery is done to re-route, or “bypass,” blood around afflicted or clogged arteries. It also improves the supply of blood and oxygen to the heart muscle. In this case, the stenosis may occur in the transplanted blood vessel. Like other stenosed arteries, they may need angioplasty or atherectomy to reopen them.

This restenosis is mainly caused by cellulofibrous hyperplasia of the intima due to migration of smooth muscle cells from the media to the intima upon stimulation by various factors (such as platelet-derived growth factor, thrombin, etc.) resulting from platelet aggregation and blood clotting at the site of injury by PTCA and the subsequent proliferation of the smooth muscle cells in the intima.

According to the American Heart Association's Heart Disease and Stroke Statistics—2003 Update, 1,025,000 angioplasties were done in the United States in 2000. Of these 561,000 were percutaneous transluminal coronary angioplasties (PTCAs); 655,000 men and 370,000 women had angioplasties, 519,000 cardiac revascularizations (also known as coronary artery bypass graft or CABG operations) were done in the United States in 2000; and CABG was performed on 371,000 men and 148,000 women.

To prevent restenosis, various drugs including anticoagulants such as heparin, etc., platelet aggregation inhibitors such as aspirin, dipyridamole, ticlopidine, prostacyclin and its derivatives, etc., cell proliferation inhibitors such as ketanserin, and antilipidemics such as eicosapentaenoic acid, lovastatin, etc. have been tested preclinically or clinically, however none of these have proved to be sufficiently effective from the clinical viewpoint American Heart Journal, 117, 777-782 (1989); ibid., 119, 232 (1990); ibid., 122, 171-187 (1991); Circulation, 81, 1753-1761 (1990); Lancet, 177-181 (1989).

With heparin, in particular, it has been reported that its long-term use (10,000 units/day, s.c.) following coronary angioplasty resulted in coronary restenosis in 82% of the cases, a very high figure as compared with control, i.e., restenosis value: 33%, with abnormal hemorrhage supervening in 41% (Journal of American College of Cardiology, 17 (2), 181A, (1991)). This augmentation of bleeding tendency has been cited as one of the adverse effects of heparin, and heparin may cause not only hemorrhage at the administration site but also bleeding in the gastrointestinal tract and, in serious cases, even intracranial hemorrhage, with death from massive loss of blood ensuing in the worst cases.

As mentioned above, the state of the art is that no effective vascular intimal hyperplasia-inhibitory agent, particularly no agent capable of preventing post-PTCA restenosis of the coronary arteries, has been discovered as yet. The primary object of the present invention, therefore, is to provide an highly effective vascular intimal hyperplasia-inhibitory composition.

Under the present circumstances, development of a device coating or a drug which would inhibit post-PTCA restenosis of the coronary arteries and hence intimal hyperplasia in atherosclerosis is desirable and currently needed.

### BRIEF SUMMARY OF THE INVENTION

The present invention comprises compositions and methods for preventing restenosis. In particular, the invention is directed to the prevention of post-percutaneous transluminal coronary angioplasty (PTCA) restenosis. The compositions of the present invention comprise one or more polysaccharides in combination with one or more pharmaceutical agents.

These compositions can be used to coat an implantable device. The methods of the instant invention comprise the coating of an angioplastic device (such as a cardiac stent). Methods of the present invention include the administration of one or more pharmacological agents in combination with one or more polysaccharides to a subject in need thereof, wherein the pharmaceutical preparation including the pharmacological agent and polysaccharide has reduced toxicity. In one aspect, the polysaccharide component is selected from group consisting of galactomannans, arabinogalactan, rhamnogalacturonan, carrageenan, and the Locust Bean Gum.

In another embodiment of the present invention, compositions and methods for preventing, for example, post-PTCA, restenosis is disclosed. These methods provide one or more compositions comprising a sufficient amount of polysaccharide and chemotherapeutic agent, optionally, an anti-proliferative or anti-angiogenesis agent can be included as well.

### DETAILED DESCRIPTION OF THE INVENTION

The present invention comprises compositions and methods for preventing restenosis. In particular, the invention is directed to the prevention of post-percutaneous transluminal coronary angioplasty (PTCA) restenosis. The compositions of the present invention comprise one or more polysaccharides in combination with one or more pharmaceutical agents.

The methods of the instant invention comprise the coating of an implantable device, or the administration, with the implant of the device, of one or more pharmacological agents in combination with one or more polysaccharides to a subject in need thereof, wherein the pharmaceutical preparation including the pharmacological agent and polysaccharide has reduced toxicity.

In one aspect, the compositions of the present invention has target delivery capability to prevent post-PTCA restenosis.

Terminology:

The following terms shall have the meaning ascribed to them herein and will be applicable throughout this document unless otherwise indicated.

“Anti-cancer drugs” are chemicals that effectively hinder growth or proliferation of cells including such molecules designated as cytotoxic, anti-metabolite, anti-proliferation, anti-angiogenic, antitumour antibiotic, alkylating agent, mitotic inhibitor, endocrine anti-hormone, biological response modifier, tumor specific monoclonal antibody, apoptosis triggering agents and other molecules that effect cell viability.

“Depolymerization” refers to partial or complete hydrolysis of the polysaccharide backbone occurring, for example, when the polysaccharide is treated chemically or enzymatically resulting in fragments of reduced size when compared with the original polysaccharide.

“Effective (or therapeutic) dose” refers to a dose of an agent that improves and/or eliminates the symptoms of the subject or the longevity of the subject suffering from or at high risk of suffering from cancer.

“Saccharide” refers to any simple carbohydrate including monosaccharides, monosaccharide derivatives, monosaccharide analogs, sugars, including those which form the individual units in an oligosaccharide or a polysaccharide.

“Monosaccharide” refers to a single unit of polyhydroxyaldehyde (aldose) or polyhydroxyketone (ketose) and derivatives and analogs thereof.

“Oligosaccharide” refers to a linear or branched chain of monosaccharides that includes up to about 20 saccharide units linked via glycosidic bonds.

“Polysaccharide” refers to polymers formed from about 10 to about 10,000 and more saccharide units linked to each other by hemiacetal or glycosidic bonds. The polysaccharide may be either a straight chain, singly branched, or multiply branched wherein each branch may have additional secondary branches, and the monosaccharides may be standard D- or L-cyclic sugars in the pyranose (6-membered ring) or furanose (5-membered ring) forms such as D-fructose and D-galactose, respectively, or they may be cyclic sugar derivatives, for example amino sugars such as D-glucosamine, deoxy sugars such as D-fucose or L-rhamnose, sugar phosphates such as D-ribose-5-phosphate, sugar acids such as D-galacturonic acid, or multi-derivatized sugars such as N-acetyl-D-glucosamine, N-acetylneuraminic acid (sialic acid), or N-sulfato-D-glucosamine.

“Backbone” means the major chain of a polysaccharide, or the chain originating from the major chain of a starting polysaccharide, having saccharide moieties sequentially linked by either α or β glycosidic bonds.

“Esterification” refers to the presence of methylesters or other ester groups at the carboxylic acid position of the uronic acid moieties of a saccharide.

“Substantially de-esterified” means, for the purposes of this application, that the degree of esterification on the backbone of the polysaccharide is less than about 5%.

“Substantially lacks secondary branches of saccharides” means that the polysaccharide backbone has less than about 1-2 secondary branches per repeating unit and no tertiary branches.

“Ligand” refers to a molecule that binds to another molecule, used especially to refer to a small molecule that binds specifically to a larger molecule, e.g., an antigen binding to an antibody, a hormone or neurotransmitter binding to a receptor, a substrate or allosteric effector binding to enzyme or receptor. For the purposes of this application, the carbohydrates that specifically bind to glyco-receptors on tumor cells are defined as “ligand”.

“Multivalent ligand binding polysaccharide” is polysaccharide that possesses two or more ligand structures which will facilitate multiple binding sites per one polymer. Due to the multivalent receptor sites on the tumor cells the multivalent ligand binding will enable a stronger and more specific interaction between the polysaccharide and the tumor.

“Glyco-receptors” refer to membrane-associated structures on cells exposed to the exterior of the cells and specifically bind carbohydrate molecules. In a particular sense, glyco-receptors refer mainly to proteins associated with tumor cells and have been described in the literature as having high affinity to carbohydrate moieties, specifically the “galactins” which have high specific binding to galactose.

“Molecular Weight” refers to molecular weight as determined by size exclusion chromatography refractive index (SEC-RI) and Multi Angle Laser Light Scattering (MALLS) which measures the dimension and absolute size of polymers.

One embodiment of the present invention is directed to compositions and methods for preventing restenosis. The compositions of the present invention comprise one or more polysaccharides in combination with one or more pharmaceutical agents. In one aspect, the polysaccharide component is selected from group consisting of galactomannans (from, e.g., Cyamopsis tetragonolobus), Arabinogalactan (from, e.g., Larix occidentalis), Rhamnogalacturonan (from, e.g., potato), Carrageenan (from, e.g., Eucheuma Seaweed), and the Locust Bean Gum (from, e.g., Ceratonia siliqua). In one aspect, the polysaccharide is a galactomannan which is available from a number of plant and microbial sources. In another aspect, an effective dose of this pharmaceutical composition can be administered to a patient in an acceptable manner well known to those skilled in the art, wherein the patient has an implantable device such as an angioplastic device.

Another embodiment of the present invention is directed to compositions and methods for preventing post-PTCA restenosis. In this embodiment, an admixture comprising an effective amount of polysaccharide and chemotherapeutic agent in a ratio suitable for reducing the toxic side-effects in a subject while being effective for preventing post-PTCA restenosis is administered to a patient in need thereof. The toxic side-effects being defined as those physiological effects (symptoms) realized by the subject resulting from the administration of the chemotherapeutic agent absent the polysaccharide.

In one aspect, a combination in the formulation comprises, an effective amount (or dose) of one or more polysaccharides and one or more chemotherapeutic/pharmaceutical, antiinflamation or anti-angiogenesis agents in a ratio suitable for preventing post-PTCA restenosis. In a particular aspect, the polysaccharide to chemotherapy ratio could be in the range from about 10:1 up to about 1:10—with approximately 2 kDa to about 300 kDa, for example, galactomannan, the optimum ratio is in the range from 6:1 to 1:3.

In another aspect, the combination comprises an effective amount of one or more polysaccharides and one or more chemotherapeutic, anti inflammation and/or anti-angiogenesis agents in a ratio suitable for effectively preventing restenosis, in particular post-PTCA, as well as reducing any potential toxic side-effect(s).

In still a further aspect of the invention, a method is provided for preventing post-PTCA restenosis. A subject in need thereof is administrated an effective dose of a composition comprising of one or more polysaccharides and is one or more chemotherapeutic agents formulated so that the chemotherapeutic, anti inflammation and/or anti-angiogenesis agents combination with the polysaccharide has sustained released and enhanced therapeutic efficacy.

The present invention relates to a polysaccharide that can be obtained by an enzymatic or chemical modification, which may reversibly interact with an anti-cancer chemotherapeutic agent and effectuate sustained release with the polysaccharide itself to prevent restenosis, e.g., post-percutaneous transluminal coronary angioplasty (PTCA) restenosis (improving the pharmacological index of PTCA as compared to that of current treatment). In accordance with the present invention, the polysaccharide comprises a polysaccharide backbone being less than about 5% esterified and containing repeating units wherein each repeating unit has a plurality of uronic acid or other glycosidic acid residues with each repeating sequence unit having at least one neutral monosaccharide residue attached thereto; at least one side chain of oligosaccharide attached to the backbone via a glucosidic bond to the neutral monosaccharide, further comprising a plurality of neutral saccharides or saccharide derivatives with the majority terminal galactose unit and an average molecular weight in the range of about 5 kDa to about 200 kDa. The polysaccharides three dimensional structure has a combination of hydrophobic, hydrophilic and slightly negative charged moieties which may interact with anti-prolific or anti-angiogenic agents and effectively deliver them to surrounding tissue as to prevent post-percutaneous transluminal coronary angioplasty (PTCA) restenosis.

Without wishing to be bound by theory, the mechanism of action may involve lectin receptors together with sustained release of pharmaceutical agents, thus preventing inflammation and restenosis. The agents may be targeted to locations via the polysaccharide component that has a certain affinity for lectin receptors.

Another embodiment of the present invention is directed to compositions and methods for preventing post-PTCA restenosis. In this embodiment, a composition comprising an effective amount of polysaccharide and chemotherapeutic, anti-proliferitic and/or anti-angiogenesis agents in a ratio suitable for reducing the toxic side-effects in a subject while being effective for preventing post-PTCA restenosis is provided. In one aspect, the polysaccharide is selected from group consisting of sulfated polysaccharide. This polysaccharide can be obtained, for example, from chemical or enzymatic depolymerization of the sea cucumber polysaccharide with a molecular weight of 2 kDa to 100 kDa and comprises galactosamine at 10 to 30%, glucuronic acid 10 to 30%, fucose 10 to 30% and total sulfate group 20 to 50% by weight. The toxic side-effects being defined as those physiological effects (symptoms) realized by the subject resulting from the administration of the chemotherapeutic agent absent the polysaccharide.

A method for inhibiting vascular intimal hyperplasia comprising administering to a patient in need thereof an effective amount of a sulfated polysaccharide is disclosed. In one aspect, the sulfated polysaccharide includes, but is not limited to, chondroitin sulfate, heparins sulfate or galactosaminglucuronate sulfate from a sea cucumber, which sulfated polysaccharide is also referred to as FGAG polysaccharide.

Chemotherapeutic agents of the present invention that are embedded in or on a polysaccharide that inhibit the proliferation of cells, for example, Aminoglutethimide, Amsacrine Anastrozole, Asparaginase, Bicalutamide, Bleomycin, Buserelin, Busulfan, Capecitabine, Carboplatin, Carmustine, Chlorambucil, Cisplatin, Cladribine, Clodronate, Cyclophosphamide, Cyproterone, Cytarabine, Dacarbazine, Dactinomycin, Daunorubicin, dexamethasone, Diethylstilbestrol, Docetaxel, Doxorubicin, Epirubicin, Estramustine, Etoposide, Exemestane, Filgrastim, Fludarabine, Fludrocortisone, Fluorouracil, Fluoxymesterone, Flutamide, Gemcitabine, Goserelin, Hydroxyurea, Idarubicin, Ifosfamide, Imatinib, Interferon-α, Irinotecan, Letrozole, Leucovorin, Leuprolide, Levamisole, Lomustine, Mechlorethamine, Medroxyprogesterone, Megestrol, Melphalan, Mercaptopurine, Mesna, methamycins, Methotrexate, Mitomycin, Mitotane, Mitoxantrone, Nilutamide, Octreotide, Oxaliplatin, Paclitaxel, Pamidronate, Pentostatin, Plicamycin, Porfimer, Procarbazine, Raltitrexed, Rituximab, Streptozocin, Tamoxifen, Temozolomide, Teniposide, Testosterone, Thioguanine, Thiotepa, Topotecan, Trastuzumab, Tretinoin, Vinblastine, Vincristine, Vindesine, Vinorelbine, daunomycin, doxorubicin and combinations thereof.

In the present invention, active derivatives of anti-proliferation agents are to be embedded in a polysaccharides matrix. This complex is designed to prevent post-PTCA restenosis, for example, taxoid is understood to mean those compounds that include paclitaxels and docetaxel, and other chemicals that have the taxane skeleton (Cortes and Pazdur, 1995, the entire teaching of which is incorporated herein by reference), and may be isolated from natural sources such as the Yew tree, or from cell culture, or chemically synthesized molecules, and in one aspect the taxane is a chemical of the general chemical formula, C₄₇H₅₁No₁₄,C₄₇H₅₁NO₁₄, including [2aR-[2aα,4.β.,4α.,β.6.β,9.α.(αR*,β.S*), 11α., 12α., 12Aα., 12α.,]]-β-(Benzoylamino)-α-hydroxybenzene propanoic acid 6,12b,bis(acetyloxy)-12-(benzoyloxy)-2a,3,4,4a,5,6,9,10,11,12,12a, 12b-dode cahydro4,11-dihydroxy4a,8,13,13-tetramethyl-5-oxo-7,11-methano-1H-cyclodeca [3,4]benz-[1,2-b]oxet-9-yl ester. It is understood that paclitaxel and docetaxel are each more effective than the other against certain types of tumors, and that in the practice of the present invention, those tumors that are more susceptible to a particular taxoid would be treated with that water soluble taxoid or taxane conjugate.

Any of the identified compounds of the present invention can be administered to a subject, including a human, by itself, or in pharmaceutical compositions where it is mixed with suitable carriers or excipients at doses therapeutically effective to prevent, treat or ameliorate a variety of disorders, including those characterized by that outlined herein. A therapeutically effective dose further refers to that amount of the compound sufficient result in the prevention or amelioration of symptoms associated with such disorders. Techniques for formulation and administration of the compounds of the instant invention may be found in Goodman and Gilman's The Pharmacological Basis of Therapeutics, Pergamon Press, latest edition.

The compounds of the present invention can be targeted to specific sites by direct injection into those sites. Compounds designed for use in the central nervous system should be able to cross the blood-brain barrier or be suitable for administration by localized injection.

Pharmaceutical compositions suitable for use in the present invention include compositions wherein the active ingredients are contained in an effective amount to achieve its intended purpose. More specifically, a therapeutically effective amount means an amount effective to prevent development of or alleviate the existing symptoms and underlying pathology of the subject being treating. Determination of the effective amounts is well within the capability of those skilled in the art.

For any compound used in the methods of the present invention, the therapeutically effective dose can be estimated initially from cell culture assays. For example, a dose can be formulated in animal models to achieve a circulating concentration range that includes the IC₅₀ (the dose where 50% of the cells show the desired effects) as determined in cell culture. Such information can be used to more accurately determine useful doses in humans.

A therapeutically effective dose refers to that amount of the compound that results in the attenuation of symptoms or a prolongation of survival in a subject. Toxicity and therapeutic efficacy of such compounds can be determined by standard pharmaceutical procedures in cell cultures or experimental animals, e.g., for determining the LD₅₀ (the dose lethal to 50% of a given population) and the ED₅₀ (the dose therapeutically effective in 50% of a given population). The dose ratio between toxic and therapeutic effects is the therapeutic index and it can be expressed as the ratio between LD₅₀ and ED₅₀. Compounds which exhibit high therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage can vary within this range depending upon the dosage form employed and the route of administration utilized. The exact formulation, route of administration and dosage can be chosen by the individual physician in view of a patient's condition. Dosage amount and interval can be adjusted individually to provide plasma levels of the active moiety which are sufficient to maintain the desired effects.

In case of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The pharmaceutical compositions of the present invention can be manufactured in a manner that is itself known, e.g., by means of conventional mixing, dissolving, granulating, levigating, emulsifying, encapsulating, entrapping or lyophilizing processes.

Pharmaceutical compositions for use in accordance with the present invention thus can be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention can be formulated in aqueous solutions, preferably in physiologically compatible buffers such as Hank's solution, Ringer's solution, or physiological saline buffer. For transmucosal administration, penetrants appropriate to the barriers to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carboxymethylcellulose, and/or polyvinylpyrrolidone (PVP). If desired, disintegrating agents can be added, such as the cross-linked polyvinyl pyrrolidone, agar, or alginic acid or a salt thereof such as sodium alginate.

Dragee cores are provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the compositions can take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodi-fluoromethane, trichlorofluoromethane, dichlorotetrafluoromethane, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated containing a powder mix of the compound and a suitable powder base such as lactose or starch.

The compounds can be formulated for parenteral administration by injection, e.g., by bolus injection or continuous infusion. Formulations for injection can be presented in unit dosage for, e.g., in ampoules or in multidose containers, with an added preservatives. The compositions can take such forms as suspensions, solutions or emulsions in oily or aqueous vehicles, and can contain formulatory agents such as suspending, stabilizing and/or dispersing agents.

Pharmaceutical formulations for parenteral administration include aqueous solutions of the active compounds in water-soluble form. Additionally, suspensions of the active compounds can be prepared as appropriate oily injection suspension. Suitable lipohilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. Optionally, the suspension can also contain suitable stabilizers or agents which increase the solubility of the compounds to allow for the preparation of highly concentrated solutions.

Alternatively, the active ingredient can be in powder form for constitution with a suitable vehicle, e.g., sterile pyrogen-free water, before use.

The compounds can also be formulated in rectal compositions such as suppositories or retention enemas, e.g., containing conventional suppository bases such as cocoa butter or other glycerides.

In addition to the formulations previously described, the compounds can also be formulated as a depot preparation. Such long acting formulations can be administered by implantation (e.g., subcutaneously or intramuscularly) or by intramuscular injection. Thus, for example, the compounds can be formulated with suitable polymeric or hydrophobic materials (e.g., as an emulsion in an acceptable oil) or ion exchange resins, or as sparingly soluble derivatives, e.g., as a sparingly soluble salt.

A pharmaceutical carrier for the hydrophobic compounds of the invention is a co-solvent system comprising benzyl alcohol, a non-polar surfactant, a water-miscible organic polymer, and an aqueous phase. Naturally, the proportions of a co-solvent system can be varied considerably without destroying its solubility and toxicity characteristics. Furthermore, the identity of the co-solvent components can be varied.

Alternatively, other delivery systems for hydrophobic pharmaceutical compounds can be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds can be delivered using a sustained-release system, such as semipermeable matrices of solid hydrophobic polymers containing the therapeutic agent. Various of sustained-release materials have been established and are well known to those skilled in the art. Sustained-release capsules can, depending on their chemical nature, release the compounds for a few weeks up to over 100 days. Depending on the chemical nature and the biological stability of the therapeutic reagent, additional strategies for protein stabilization can be employed.

The pharmaceutical compositions also can comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Many of the compounds of the invention can be provided as salts with pharmaceutically compatible counterions. Pharmaceutically compatible salts can be formed with many acids, including but not limited to hydrochloric, sulfuric, acetic, lactic, tartaric, malic, succinic, etc. Salts tend to be more soluble in aqueous or other protonic solvents that are the corresponding free base forms.

Suitable routes of administration can, e.g., include oral, rectal, transmucosal, transdermal, or intestinal administration; parenteral delivery, including intramuscular, subcutaneous, intramedullary injections, as well as intrathecal, direct intraventricular, intravenous, intraperitoneal, intranasal, or intraocular injections.

Alternatively, one can administer the compound in a local rather than systemic manner, e.g., via injection of the compound directly into an affected area, often in a depot or sustained release formulation.

Furthermore, one can administer the compound in a targeted drug delivery system, e.g., in a liposome coated with an antibody specific for affected cells. The liposomes will be targeted to and taken up selectively by the cells.

The compositions can, if desired, be presented in a pack or dispenser device which can contain one or more unit dosage forms containing the active ingredient. The pack can, e.g., comprise metal or plastic foil, such as a blister pack. The pack or dispenser device can be accompanied by instruction for administration. Compositions comprising a compound of the invention formulated in a compatible pharmaceutical carrier can also be prepared, placed in an appropriate container, and labeled for treatment of an indicated condition. Suitable conditions indicated on the label can include treatment of a disease such as described herein.

Assay methods:

1. Smooth Muscle Cell Proliferation-Inhibitory Action

The medial smooth muscle layer was excised from the rat thoracic aorta and incubated by the explant method (J. Cell. Physiol., 142, 342, 1990, the entire teaching of which is incorporated herein by reference). This smooth muscle cell culture was adjusted to 2×10³ cells/200 μL culture fluid. On the next day, DHG-1, dissolved in a small amount of the culture fluid was added to a final concentration of 0 to 1.0 μg/mL and, after 5 days of incubation, viable cells were counted by the MTT method (J. Immunol. Methods, 65, 55, 1983 the entire teaching of which is incorporated herein-by references) and the inhibition rate (%) was calculated.

The combinational therapy should inhibited the proliferation of vascular smooth muscle cells at concentrations at lower than 0.1 μg/mL.

2. Smooth muscle cell migration-inhibitory action

A rat smooth muscle cell culture was prepared, DHG-1 was added to a final concentration of 0 to 10 μg/mL and 250 μL of the mixture was placed in the upper chamber of a two-layer culture plate. A solution (600 μL) of platelet-derived growth factor (PDGF) showing chemotaxis at 1 ng/mL and DHG-1 (final concentration 0 to 10 μg/mL) was introduced into the lower chamber. The boundary between the upper and lower chambers was made of a filter having a number of small openings allowing passage of smooth muscle cells. After allowing cell migration from the upper to the lower chamber for 6 hours, the contents of the lower chamber alone were incubated for 24 hours and, after fixation and staining the cells were counted and the inhibition rate (%) was calculated. At concentrations of 1.0 μg/mL, the polysaccharide combination with paclitaxel should inhibited the migration of vascular smooth muscle cells in a dose-dependent manner.

The results of Pharmacological Tests may provided evidence for the post-PTCA restenosis-inhibitory action of the composition of polysaccharide and for example paclitaxel inhibit migration or proliferation of medial smooth muscle cells.

The composition of polysaccharide, and chemotherapeutic, anti-proliferation, anti-inflammation or anti-angiogenesis are active in vascular intimal hyperplasia-inhibition by inhibiting for example the migration or proliferation of vascular smooth muscle cells, without the hemorrhagic symptom causes by heparin.

1. A composition for preventing restenosis, comprising one or more polysaccharides in combination with one or more pharmaceutical agents.

2. The composition of claim 1, wherein said polysaccharide is selected from the group consisting of galactomannan, arabinogalactan, rhamnogalacturon, carrageenan, locust bean gum and combinations thereof.

3. The composition of claim 2, wherein said polysaccharide is galactomannan.

4. The composition of claim 1, wherein said pharmaceutical agent is an anti-proliferative, anti-angiogenic, anti-cancer agent or combinations thereof.

5. The composition of claim 4, wherein said anti-cancer agent is selected from the group consisting of Aminoglutethimide, Amsacrine Anastrozole, Asparaginase, Bicalutamide, Bleomycin, Buserelin, Busulfan, Capecitabine, Carboplatin, Carmustine, Chlorambucil, Cisplatin, Cladribine, Clodronate, Cyclophosphamide, Cyproterone, Cytarabine, Dacarbazine, Dactinomycin, Daunorubicin, dexamethasone, Diethylstilbestrol, Docetaxel, Doxorubicin, Epirubicin, Estramustine, Etoposide, Exemestane, Filgrastim, Fludarabine, Fludrocortisone, Fluorouracil, Fluoxymesterone, Flutamide, Gemcitabine, Goserelin, Hydroxyurea, Idarubicin, Ifosfamide, Imatinib, Interferon-α, Irinotecan, Letrozole, Leucovorin, Leuprolide, Levamisole, Lomustine, Mechlorethamine, Medroxyprogesterone, Megestrol, Melphalan, Mercaptopurine, Mesna, methamycins, Methotrexate, Mitomycin, Mitotane, Mitoxantrone, Nilutamide, Octreotide, Oxaliplatin, Paclitaxel, Pamidronate, Pentostatin, Plicamycin, Porfimer, Procarbazine, Raltitrexed, Rituximab, Streptozocin, Tamoxifen, Temozolomide, Teniposide, Testosterone, Thioguanine, Thiotepa, Topotecan, Trastuzumab, Tretinoin, Vinblastine, Vincristine, Vindesine, Vinorelbine, daunomycin, doxorubicin, and combinations thereof.

6. The composition of claim 4, wherein said anti-proliferative agent is taxane.

7. The composition of claim 6, wherein said taxane is selected from the group consisting of C₄₇H₅₁No₁₄,C₄₇H₅₁NO₁₄, including [2aR-[2α,4.β.,4α.,β.6.β,9. α(αR*,β.S*), 11α.,12α.,12Aα.,12α.,]]-β-(Benzoylamino)-α-hydroxybenzene propanoic acid 6,12b,bis(acetyloxy)-12-(benzoyloxy)-2a,3,4,4a,5,6,9,10,11,12,12a, 12b-dode cahydro4,11-dihydroxy4a,8,13,13-tetramethyl-5-oxo-7,11-methano-1H-cyclodeca [3,4]benz-[1,2-b]oxet-9-yl ester and combinations thereof.

8. The composition of claim 1, wherein said restenosis is percutaneous transluminal coronary angioplasties.

9. An implantable device comprising a surface coated with a composition having one or more polysaccharides and one or more pharmaceutical agents.

10. The device of claim 9, wherein said polysaccharide is selected from the group consisting of galactomannan, arabinogalactan, rhamnogalacturon, carrageenan, locust bean gum and combinations thereof.

11. The device of claim 10, wherein said polysaccharide is galactomannan.

12. The device of claim 9, wherein said pharmaceutical agent is an anti-proliferative, anti-angiogenic, anti-cancer agent or combinations thereof.

13. The device of claim 9, wherein said device is an angioplastic device.

14. The device of claim 13, wherein said angioplastic device is a cardiac stent.

15. A method for preventing restenosis, comprising administering to a subject in need a composition having one or more polysaccharides in combination with one or more pharmaceutical agents, wherein said combination has reduced toxicity.

16. The method of claim 15, wherein said polysaccharide is selected from the group consisting of galactomannan, arabinogalactan, rhamnogalacturon, carrageenan, locust bean gum and combinations thereof.

17. The method of claim 16, wherein said polysaccharide is galactomannan.

18. The method of claim 15, wherein said pharmaceutical agent is an anti-proliferative, anti-angiogenic, anti-cancer agent or combinations thereof.

19. The method of claim 18, wherein said anti-cancer agent is selected from the group consisting of Aminoglutethimide, Amsacrine Anastrozole, Asparaginase, Bicalutamide, Bleomycin, Buserelin, Busulfan, Capecitabine, Carboplatin, Carmustine, Chlorambucil, Cisplatin, Cladribine, Clodronate, Cyclophosphamide, Cyproterone, Cytarabine, Dacarbazine, Dactinomycin, Daunorubicin, dexamethasone, Diethylstilbestrol, Docetaxel, Doxorubicin, Epirubicin, Estramustine, Etoposide, Exemestane, Filgrastim, Fludarabine, Fludrocortisone, Fluorouracil, Fluoxymesterone, Flutamide, Gemcitabine, Goserelin, Hydroxyurea, Idarubicin, Ifosfamide, Imatinib, Interferon-α, Irinotecan, Letrozole, Leucovorin, Leuprolide, Levamisole, Lomustine, Mechlorethamine, Medroxyprogesterone, Megestrol, Melphalan, Mercaptopurine, Mesna, methamycins, Methotrexate, Mitomycin, Mitotane, Mitoxantrone, Nilutamide, Octreotide, Oxaliplatin, Paclitaxel, Pamidronate, Pentostatin, Plicamycin, Porfimer, Procarbazine, Raltitrexed, Rituximab, Streptozocin, Tamoxifen, Temozolomide, Teniposide, Testosterone, Thioguanine, Thiotepa, Topotecan, Trastuzumab, Tretinoin, Vinblastine, Vincristine, Vindesine, Vinorelbine, daunomycin, doxorubicin, and combinations thereof.

20. The method of claim 18, wherein said anti-proliferative agent is taxane.

21. The method of claim 20, wherein said taxane is selected from the group consisting of C₄₇H₅₁,No₁₄,C₄₇H₅₁NO₁₄, including [2aR-[2aα,4.β.,4α.,β.6.β,9.α(αR*,β.S*), 11α., 12α., 12Aα., 12α.,]]-β-(Benzoylamino)-α-hydroxybenzene propanoic acid 6,12b,bis(acetyloxy)-12-(benzoyloxy)-2a,3,4,4a,5,6,9,10,11,12,12a, 12b-dode cahydro4,11-dihydroxy4a,8,13,13-tetramethyl-5-oxo-7,11-methano-1H-cyclodeca [3,4]benz-[1,2-b]oxet-9-yl ester and combinations thereof.

22. The method of claim 15, wherein said restenosis is percutaneous transluminal coronary angioplasties.

23. The method of claim 15, wherein said administration of said polysaccharide and pharmaceutical agent are in a ration of about 10:1 to about 1:10 polysaccharide to pharmaceutical agent.

24. The method of claim 15, wherein said polysaccharide has a molecular weight ranging from about 2 kDa to about 300 kDa.

25. The method of claim 15, wherein said polysaccharide is a sulfated polysaccharide.

26. The method of claim 25, wherein said sulfated polysaccharide is selected from the group consisting of chondroitin sulfate, heparin sulfate, galactosaminglucuronate sulfate and a combination thereof.
